(19) United States
(12) Patent Application Publication
(10) Pub. No.: US 2026/0221230 A1
(43) Pub. Date: Jul. 30, 2026

(54) MACHINE LEARNING BASED OPTIMIZATION OF THERAPEUTIC PROTEINS

(71) Applicant: Genentech, Inc., South San Francisco, CA (US)

(72) Inventors: Samuel Don STANTON, Brooklyn, NY (US); Amy WANG, Foster City, CA (US); Andrew Martin WATKINS, San Francisco, CA (US)

(21) Appl. No.: 19/464,502

(22) Filed: Jan. 29, 2026

Related U.S. Application Data
(60) Provisional application No. 63/751,696, filed on Jan. 30, 2025.

(51) Int. Cl. G16B 40/00; G06F 30/27

(52) U.S. Cl. CPC G16B 40/00; G06F 30/27

(57) ABSTRACT

A method may include receiving a candidate protein molecule. A property computation model is applied to determine, based on an amino acid residue sequence of the candidate protein molecule, one or more biophysical descriptors of the candidate protein molecule. The one or more biophysical descriptors define one or more surface properties approximating one or more developability traits of the candidate protein molecule. The candidate protein molecule is screened based on the one or more biophysical descriptors of the candidate protein molecule. Alternatively, the generating of one or more additional candidate protein molecules, for example, by modifying the amino acid residue sequence of the candidate protein molecule, is guided by the one or more biophysical descriptors of the candidate protein molecule. Related systems and computer program products are also provided.

### CROSS REFERENCE TO RELATED APPLICATION

This application claims priority to U.S. Provisional Application No. 63/751,696, entitled “MACHINE LEARNING BASED OPTIMIZATION OF THERAPEUTIC PROTEINS” and filed on Jan. 30, 2025, the disclosure of which is incorporated herein by reference in its entirety.

### TECHNICAL FIELD

The subject matter described herein relates generally to molecular design, and more specifically, to machine learning enabled design of therapeutic protein molecules with biophysical property improvement.

### INTRODUCTION

A molecule is a group of two more atoms held together by chemical bonds. Molecules form the smallest identifiable unit into which a pure substance can be divided while still retaining the composition and chemical properties of that substance. Various properties of a molecule, including its ability to function as a therapeutic, may be contingent upon its composition and conformation (or three-dimensional structure). Large molecules (also known as biopharmaceuticals, biologicals, or biologics) are molecules ranging between approximately 3000 Daltons and 150,000 Daltons in molecular weight. Large molecule drugs are often derivatives of natural human proteins, which modulate many essential cellular functions such as enzymatic reactions, transport of molecules, regulation and execution of a number of biological pathways, cell growth, proliferation, nutrient uptake, morphology, motility, intercellular communication, and/or the like. Examples of therapeutic proteins include antibodies, chimeric antigen receptors (CARs), enzymes, hormones, cytokines, and/or the like. A single large molecule can have more than 1,300 amino acid residues, which are linked by peptide bonds to form one or more polypeptide. Due to their size and complexity, large molecule drugs are recombinantly produced by engineered cells instead of being chemically synthesized like the majority of small molecule drugs. Moreover, large molecule therapeutics are usually delivered through injection or infusion due to the ineffectiveness of oral administration. The development of a large molecule drug may entail designing one or more sequences of amino acid residues capable of binding to a target (e.g., a protein, a nucleic acid, and/or the like) with sufficient specificity and absent undesirable traits such as immunogenicity, self-association, instability, and/or the like.

### SUMMARY

Systems, methods, and articles of manufacture, including computer program products, are provided for machine learning enabled design of therapeutic protein molecules with biophysical property improvement. In the context of therapeutic protein design, developability refer to an assessment of the likelihood of a candidate protein molecule becoming a successful, manufacturable, stable, and safe drug. Even when a candidate protein molecule exhibits adequate binding affinity and specificity toward a target molecule (e.g., a viral antigen, a tumor antigen, and/or the like), the presence of developability liabilities, such as suboptimal solubility, stability, viscosity, aggregation, immunogenicity, and expression, can disqualify the candidate protein molecule from further development. In some cases, the occurrence of late-stage failures may be avoided by screening candidate protein molecules for the presence of developability liabilities, including for developability liabilities that may be present in computationally (or partially computationally) designed protein sequences. In some cases, candidate protein molecules may be screened for the presence of developability liabilities based on biophysical descriptors defining one or more surface properties. For example, in some cases, one or more surface properties of interest, such as those associated with developability, may be defined by one or more corresponding biophysical descriptors for electrostatics, hydrophobicity, chemical liabilities, and/or the like. In some cases, a property computation model may be trained to determine, based at least on an amino acid residue sequence of a candidate protein molecule, the one or more biophysical descriptors defining the surface properties associated with developability. In some cases, the candidate protein molecule may be excluded from further development where the one or more biophysical descriptors of the candidate protein molecule indicate the presence of developability liabilities. Alternatively and/or additionally, the candidate protein molecule may be improved, for example, by modifying the corresponding amino acid residue sequence, to improve the one or more biophysical descriptors and mitigate (or eliminate) the concomitant developability liabilities.

In one aspect, there is provide a system for machine learning enabled design of therapeutic protein molecules with biophysical property improvement. The system may include at least one data processor and at least one memory. The at least one memory may store instructions that result in operations when executed by the at least one data processor. The operations may include: receiving a candidate protein molecule, wherein the candidate protein molecule comprises an amino acid residue sequence; applying a property computation model to determine, based at least on the amino acid residue sequence of the candidate protein molecule, one or more biophysical descriptors of the candidate protein molecule, wherein the one or more biophysical descriptors define one or more surface properties of the candidate protein molecule; and screening the candidate protein molecule based at least on the one or more biophysical descriptors of the candidate protein molecule.

In another aspect, there is provided a computer-implemented method for machine learning enabled design of therapeutic protein molecules with biophysical property improvement. The method may include: receiving a candidate protein molecule, wherein the candidate protein molecule comprises an amino acid residue sequence; applying a property computation model to determine, based at least on the amino acid residue sequence of the candidate protein molecule, one or more biophysical descriptors of the candidate protein molecule, wherein the one or more biophysical descriptors define one or more surface properties of the candidate protein molecule; and screening the candidate protein molecule based at least on the one or more biophysical descriptors of the candidate protein molecule.

In another aspect, there is provided a computer program product for machine learning enabled design of therapeutic protein molecules with biophysical property improvement. The computer program product may include a non-transitory computer readable medium storing instructions that result in operations when executed by at least one data processor. The operations may include: receiving a candidate protein molecule, wherein the candidate protein molecule comprises an amino acid residue sequence; applying a property computation model to determine, based at least on the amino acid residue sequence of the candidate protein molecule, one or more biophysical descriptors of the candidate protein molecule, wherein the one or more biophysical descriptors define one or more surface properties of the candidate protein molecule; and screening the candidate protein molecule based at least on the one or more biophysical descriptors of the candidate protein molecule.

In some variations, one or more features disclosed herein including the following features can optionally be included in any feasible combination.

In some variations, the one or more biophysical descriptors define the one or more surface properties for electrostatics, hydrophobicity, and/or chemical liabilities.

In some variations, the one or more surface properties approximate one or more developability traits.

In some variations, the one or more developability traits include clearance, solubility, viscosity, storability, expression, manufacturability, polyspecificity, polyreactivity, pharmacokinetics (PK), and stability.

In some variations, the property computation model includes one or more machine learning models.

In some variations, the property computation model includes one or more ensembles of machine learning models.

In some variations, each ensemble of machine learning models includes an encoder coupled with one or more regression models.

In some variations, each ensemble of machine learning models includes a transformer encoder coupled with a convolutional encoder that is further coupled with one or more regression models.

In some variations, each ensemble of machine learning models is trained to determine an individual biophysical descriptor.

In some variations, the one or more ensembles of machine learning models includes one ensemble trained to determine a biophysical descriptor defining an electrostatic surface property.

In some variations, the one or more ensembles of machine learning models includes an additional ensemble trained to determine a biophysical descriptor defining a hydrophobicity surface property.

In some variations, the candidate protein molecule is screened based at least on whether the one or more biophysical descriptors of the candidate protein molecule satisfy one or more criteria.

In some variations, the candidate protein molecule is screened based on a plurality of biophysical descriptors.

In some variations, the screening the candidate protein molecule includes determining a utility metric quantifying an extent to which the plurality of biophysical descriptors of the candidate protein molecules improve upon a same plurality of biophysical descriptors of one or more other candidate protein molecules.

In some variations, the utility metric comprises one or more of an expected hypervolume improvement (EHVI), noisy expected hypervolume improvement (NEHVI), Pareto efficient global optimization (ParEGO), max-value entropy search method (MESMO), joint entropy search (JES), and expected multivariate rank.

In some variations, the screening the candidate protein molecule includes selecting the candidate protein molecule for synthesis and/or experimental validation.

In some variations, the screening the candidate protein molecule includes further modifying the amino acid residue sequence of the candidate protein molecule to generate one or more additional candidate protein molecules.

In some variations, a molecule design computation model is applied to further modify the amino acid residue sequence of the candidate protein molecule while guided by the one or more biophysical descriptors.

In another aspect, there is provide a system for machine learning enabled design of therapeutic protein molecules with biophysical property improvement. The system may include at least one data processor and at least one memory. The at least one memory may store instructions that result in operations when executed by the at least one data processor. The operations may include: applying a molecule design computation model to generate a candidate protein molecule; applying the molecule design computation model to generate a different candidate protein molecule; applying a property computation model to determine, based at least on an amino acid residue sequence of the candidate protein molecule, one or more biophysical descriptors defining one or more surface properties of the candidate protein molecule, wherein the one or more surface properties of the candidate protein molecule approximate one or more developability traits of the candidate protein molecule; applying the property computation model to determine, based at least on an amino acid residue sequence of the different candidate protein molecule, one or more biophysical descriptors defining one or more surface properties of the different protein molecule, wherein the one or more surface properties of the different candidate protein molecule approximate one or more developability traits of the different candidate protein molecule; determining the candidate protein molecule exhibits one or more better developability traits than the different candidate protein molecule; and applying the molecule design computation model to generate, based at least on the candidate protein molecule, one or more additional candidate protein molecules.

In another aspect, there is provided a computer-implemented method for machine learning enabled design of therapeutic protein molecules with biophysical property improvement. The method may include: applying a molecule design computation model to generate a candidate protein molecule; applying the molecule design computation model to generate a different candidate protein molecule; applying a property computation model to determine, based at least on an amino acid residue sequence of the candidate protein molecule, one or more biophysical descriptors defining one or more surface properties of the candidate protein molecule, wherein the one or more surface properties of the candidate protein molecule approximate one or more developability traits of the candidate protein molecule; applying the property computation model to determine, based at least on an amino acid residue sequence of the different candidate protein molecule, one or more biophysical descriptors defining one or more surface properties of the different protein molecule, wherein the one or more surface properties of the different candidate protein molecule approximate one or more developability traits of the different candidate protein molecule; determining the candidate protein molecule exhibits one or more better developability traits than the different candidate protein molecule; and applying the molecule design computation model to generate, based at least on the candidate protein molecule, one or more additional candidate protein molecules.

In another aspect, there is provided a computer program product for machine learning enabled design of therapeutic protein molecules with biophysical property improvement. The computer program product may include a non-transitory computer readable medium storing instructions that result in operations when executed by at least one data processor. The operations may include: applying a molecule design computation model to generate a candidate protein molecule; applying the molecule design computation model to generate a different candidate protein molecule; applying a property computation model to determine, based at least on an amino acid residue sequence of the candidate protein molecule, one or more biophysical descriptors defining one or more surface properties of the candidate protein molecule, wherein the one or more surface properties of the candidate protein molecule approximate one or more developability traits of the candidate protein molecule; applying the property computation model to determine, based at least on an amino acid residue sequence of the different candidate protein molecule, one or more biophysical descriptors defining one or more surface properties of the different protein molecule, wherein the one or more surface properties of the different candidate protein molecule approximate one or more developability traits of the different candidate protein molecule; determining the candidate protein molecule exhibits one or more better developability traits than the different candidate protein molecule; and applying the molecule design computation model to generate, based at least on the candidate protein molecule, one or more additional candidate protein molecules.

In some variations, one or more features disclosed herein including the following features can optionally be included in any feasible combination.

In some variations, the molecule design computation model generates the one or more additional candidate protein molecules by modifying the amino acid residue sequence of the candidate protein molecule.

In some variations, the one or more biophysical descriptors define one or more surface properties for electrostatics, hydrophobicity, and/or chemical liabilities.

In some variations, the one or more developability traits include clearance, solubility, viscosity, storability, expression, manufacturability, polyspecificity, polyreactivity, pharmacokinetics (PK), and stability.

In some variations, the property computation model includes one or more machine learning models.

In some variations, the property computation model includes one or more ensembles of machine learning models.

In some variations, each ensemble of machine learning models includes an encoder coupled with one or more regression models.

In some variations, each ensemble of machine learning models includes a transformer encoder coupled with a convolutional encoder that is further coupled with one or more regression models.

In some variations, each ensemble of machine learning models is trained to determine an individual biophysical descriptor.

In some variations, the one or more ensembles of machine learning models includes one ensemble trained to determine a biophysical descriptor defining an electrostatic surface property.

In some variations, the one or more ensembles of machine learning models includes an additional ensemble trained to determine a biophysical descriptor defining a hydrophobicity surface property.

In some variations, the candidate protein molecule is determined to exhibit the one or more better developability traits than the different candidate protein molecule based at least on a plurality of biophysical descriptors.

In some variations, a utility metric quantifying an extent to which the plurality of biophysical descriptors of the candidate protein molecules improve upon a same plurality of biophysical descriptors of the different candidate protein molecules is determined.

In some variations, the utility metric comprises one or more of an expected hypervolume improvement (EHVI), noisy expected hypervolume improvement (NEHVI), Pareto efficient global optimization (ParEGO), max-value entropy search method (MESMO), joint entropy search (JES), and expected multivariate rank.

In some variations, the candidate protein molecule is selected, based at least on the utility metric of the candidate protein molecule satisfying one or more criteria, for synthesis and/or experimental validation.

In some variations, the candidate protein molecule and the different candidate protein molecule each comprise at least a portion of an antibody.

In some variations, the candidate protein molecule and the different candidate protein molecule each comprise a variable region (Fv), an antigen binding fragment (Fab), and/or a complementarity determining region (CDR) of an antibody.

In some variations, the molecule design computation model is applied to continue modifying the candidate protein molecule and incrementally improve the one or more biophysical descriptors of the candidate protein molecule until one or more criteria are satisfied.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the design of protein molecules, including therapeutic proteins such as antibodies, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a protein design system, in accordance with some example embodiments; and

FIG. 2A depicts a flowchart illustrating an example of a process for machine learning enabled design of therapeutic protein molecules with biophysical property improvement, in accordance with some example embodiments;

FIG. 2B depicts a flowchart illustrating another example of a process for machine learning enabled design of therapeutic protein molecules with biophysical property improvement, in accordance with some example embodiments;

FIG. 3A depicts a schematic diagram illustrating an example deployment of a molecule design system, in accordance with some example embodiments; and

FIG. 3B depicts a schematic diagram illustrating an example deployment of a molecule design system, in accordance with some example embodiments;

FIG. 4A depicts graphs illustrating a comparison of various structurally determined surface properties and the corresponding surface properties determined using a sequence-based property computation model, in accordance with some example embodiments;

FIG. 4B depicts a graph illustrating the computational scalability of a sequence-based property computation model, in accordance with some example embodiments;

FIG. 5A depicts an illustration of a protein molecule annotated for amino acid residues targeted for modification to improve developability liabilities, in accordance with some example embodiments;

FIG. 5B depicts an illustration of another protein molecule annotated for amino acid residues targeted for modification to improve developability liabilities, in accordance with some example embodiments; and

FIG. 6 depicts a block diagram illustrating an example of a computing system, in accordance with some example embodiments.

When practical, similar reference numbers denote similar structures, features, or elements.

### DETAILED DESCRIPTION

A molecule may be designed to exhibit multiple desirable properties including, in the case of protein therapeutics, antigen binding affinity, functional activity, immunogenicity, pharmacokinetics, expression, stability, solubility, viscosity, aggregation propensity, and/or the like. Lead optimization is one variation of molecular design in which a lead molecule (or another select molecule) is modified to enhance desirable properties while minimizing undesirable ones. For example, in some cases, the lead molecule may be an antibody identified through an animal immunization campaign as having binding affinity towards a target molecule, such as a viral antigen, a tumor antigen, and/or the like. While binding affinity is an example of a desirable property, the lead molecule may also exhibit one or more undesirable properties, such as insufficient human-ness, poor expression, immunogenicity, in vivo instability, and/or the like. As is, the lead molecule is unlikely to be a viable protein therapeutic and is therefore unsuitable for further drug development efforts. Instead, the lead molecule may undergo lead optimization, which in this case may include modifying the underlying sequence of amino acid residues, for example, by changing the identity of one or more constituent amino acid residues, such that the resulting molecules exhibit better properties than the lead molecule.

Therapeutic proteins, including biologically derived macromolecules such as antibodies, hormones, enzymes, and growth factors, are a powerful class of medicine due to their specificity, efficacy, and pharmacological properties. In some cases, the development of therapeutic proteins may require improving a host of so-called “developability” factors including, for example, clearance, solubility, viscosity, storability, expression, manufacturability, polyspecificity (or polyreactivity), pharmacokinetics (PK) (e.g., long in vivo half-life for sustained efficacy), and stability (e.g., resistant to heat, pH changes, storage conditions, and/or the like). Developability factors determine the likelihood of a candidate protein molecule becoming a viable therapeutic protein that is not only effective but also safe, manufacturable, and stable. For example, even when a candidate protein molecule exhibits adequate binding affinity and specificity toward a target molecule (e.g., a viral antigen, a tumor antigen, and/or the like), the presence of developability liabilities may still prevent the candidate protein molecule from becoming a viable protein therapeutic. As such, developability properties can substantially impact the time and cost of development as well as subsequent likelihood of success in clinical trials. Due to the high material requirements of wetlab experimental testing, developability liabilities are often assessed later in the drug development pipeline, which can lead to expensive repercussions when issues arise. While substantial investments have been made towards mitigating the bottleneck imposed by developability liabilities, conventional solutions to screen candidate protein molecules with potential developability issues earlier in the drug development pipeline remain unsatisfactory.

In some example embodiments, a candidate molecule may be screened for developability liabilities based on one or more biophysical descriptors defining surface properties, such as those for electrostatics, hydrophobicity, chemical liabilities, and/or the like. In some cases, biophysical descriptors may be capable of characterizing the developability of the candidate molecule for further development at least because many functional properties of the candidate molecule are directly influenced by its underlying physical attributes. For example, in some cases, the developability of a candidate molecule can be assessed by comparing one or more surface properties to those of clinically tested molecules. For less complex small molecules, molecular descriptors can used in medicinal chemistry to inform oral drug candidates. However, widespread adoption of molecular descriptors for protein molecules (e.g., antibodies), such as molecular descriptors based on three-dimensional structure, has been thwarted by the inherent complexities of large molecule modalities. Molecular descriptors for protein molecules are not only more computationally intensive to obtain, but also exhibit significant variability depending on parametrization. Efforts towards characterizing the parameter space reached little consensus on the role of surface definitions, structure preparation, and molecular dynamics (MD). Biophysical rules can be overly reductionist and may eliminate too many viable candidate molecules if applied indiscriminately. This can be especially problematic where correlations with experimental properties are weak or if the starting set of candidate molecules is limited. Lead optimization is therefore used to resolve developability challenges but this process can be painstaking, even with significant expertise, as enhancing one property can often times worsen another. As such, filtering for molecules resembling clinical antibodies, which might be easier to develop, remains a valuable strategy for tackling the complexities with therapeutic protein design.

Various embodiments of the present disclosure overcome the limitations of existing molecular design protocols, including computational methodologies, by providing a sequence-based design framework for generating protein molecules with one or more surface properties of interest, such as those associated with increased (or optimal) developability traits or reduced (or minimal) developability liabilities. In some cases, the developability traits (or developability liabilities) of a candidate protein molecule may include clearance, solubility, viscosity, storability, expression, manufacturability, polyspecificity (or polyreactivity), pharmacokinetics (PK) (e.g., long in vivo half-life for sustained efficacy), stability (e.g., resistant to heat, pH changes, storage conditions, and/or the like), and/or the like. In some cases, one or more surface properties of interest, such as those associated with developability, may be defined by one or more biophysical descriptors for electrostatics, hydrophobicity, chemical liabilities, and/or the like. In some cases, a property computation model may be trained to determine, based at least on the amino acid residue sequence of at least a portion of a candidate protein molecule (e.g., the variable (Fv) domain of a candidate antibody), the one or more biophysical descriptors defining the surface properties associated with developability. As described in more detail below, in some cases, the generating of candidate protein molecules for further development, including the computational generation of the corresponding of amino acid residue sequences, may be guided by the biophysical descriptors determined by the property computation model. For instance, in some cases, guidance from the property computation model may enable the identification of candidate protein molecules with developability liabilities. In some cases, candidate protein molecules exhibiting developability liabilities may be excluded from synthesis, experimental validation, and other further development efforts. Alternatively, in some cases, guidance from the property computation model may enable rational modifications (e.g., rational electrostatic, hydrophobic, and/or chemical liability modifications) to reduce (or minimize) developability liabilities present in candidate protein molecules such that the candidate protein molecules that do advance to subsequent stages of drug development have a higher likelihood of becoming viable protein therapeutics. It should be appreciated that distilling structure-based biophysical descriptors into a sequence-based property computation model provides a novel solution to screening candidate protein molecules for developability liabilities that is more computationally efficient and scalable than conventional structural-based approaches that require protein folding and physics-based calculations.

In some example embodiments, the property computation model may be trained to determine, based at least on the amino acid residue sequence of at least a portion of a candidate protein molecule (e.g., the variable (Fv) domain of a candidate antibody), one or more biophysical descriptors defining the surface properties associated with developability. In some cases, the property computation model may include one or more machine learning models. For example, in some cases, the property computation model may include a regression model coupled with one or more encoders (e.g., convolutional encoder, transformer encoder, and/or the like). Moreover, in some cases, the regression model may be trained to determine, based at least on one or more features extracted from the amino acid residue sequence of the candidate protein molecule by the one or more encoders, the one or more biophysical descriptors. In some cases, the property computation model may be trained on one or more ground-truth surface properties derived from physics-based modeling of protein structures, such as those folded from the paired Observed Antibody Space (pOAS). Furthermore, in some cases, the biophysical descriptors defining the surface properties associated with developability may be benchmarked to establish robust optimization parameters and strengthen the nexus between the biophysical descriptors and experimental data.

In some example embodiments, the generating of candidate protein molecules by a molecule design computation model may be guided by the biophysical descriptors determined by the property computation model. For example, in some cases, the molecule design computation model may be a machine learning model (e.g., diffusion model and/or the like) trained on a masked token objective. In some cases, with guidance from the biophysical descriptors determined by the property computation model, the molecule design computation model may be trained to generate protein sequences (or amino acid residue sequences) whose surface properties are associated with incrementally better developability traits. For example, in some cases, the molecule design computation model may be applied to modify an input protein sequence to generate at least a first candidate protein sequence and a second candidate protein sequence. In some cases, the molecule design computation model may modify the input protein sequence by inserting, deleting, and/or changing the identity (or type) of one or more constituent amino acid residues. In some cases, the property computation model may be applied to determine, for each of the first candidate protein sequence and the second candidate protein sequence, one or more corresponding biophysical descriptors. In some cases, the molecule design computation model may be applied to further modify the first candidate protein sequence instead of the second candidate protein sequence based at least on the biophysical descriptors of the first candidate protein sequence being associated with better developability traits than the biophysical descriptors of the second candidate protein sequence. Alternatively, the first candidate protein sequence may advance to a subsequent stage of the drug development pipeline instead of the second candidate protein sequence.

In some example embodiments, the molecule design computation model may generate multiple candidate protein sequences, such as the first candidate protein sequence and the second candidate protein sequence. In some cases, the selection between two or more candidate protein sequences, such as the first candidate protein sequence and the second candidate protein sequence, may be based on multiple biophysical descriptors. Accordingly, in some cases, the two or more candidate protein sequences may undergo multi-objective optimization in which a subset of candidate protein sequences are selected based at least on a utility metric. In some cases, the utility metric may quantify the extent to which the biophysical descriptors of a candidate protein sequence improves upon those of a different candidate protein sequence from the same design iteration (or a baseline candidate protein sequence from a previous design iteration). In some cases, the utility metric may correspond to the probability of a candidate protein sequence being one of the Pareto-optimal solutions populating the Pareto frontier. In some cases, the utility metric of a candidate protein sequence may correspond to the distance (or proximity) between the candidate protein sequence and the Pareto frontier, meaning that the candidate protein sequence may be considered a Pareto-optimal solution on the Pareto frontier if its utility metric satisfies one or more thresholds. Examples of utility metrics include expected hypervolume improvement (EHVI), noisy expected hypervolume improvement (NEHVI), Pareto efficient global optimization (ParEGO), max-value entropy search method (MESMO), joint entropy search (JES), expected multivariate rank, and/or the like.

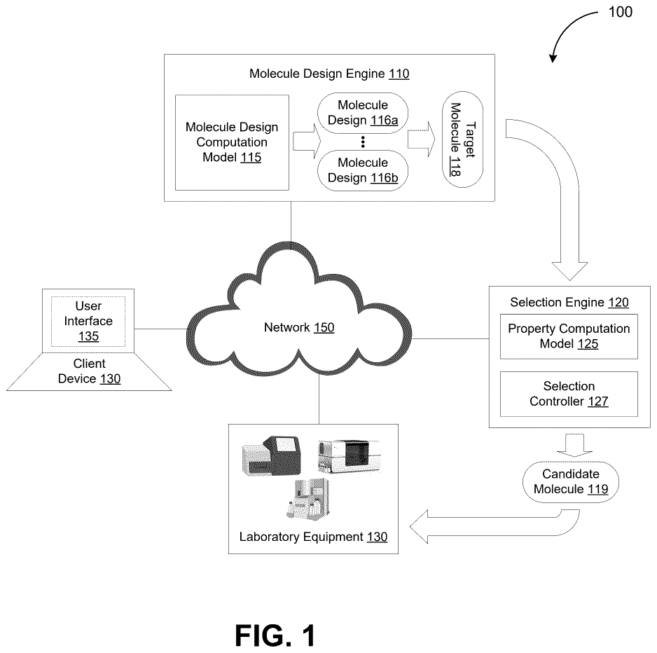

FIG. 1 depicts a system diagram illustrating an example of a protein design system 100, in accordance with some example embodiments. Referring to FIG. 1 the protein design system 110 may include a molecule design engine 110, a selection engine 120, one or more laboratory equipment 130, and a client device 140. As shown in FIG. 1, the molecule design engine 110, the selection engine 120, the one or more laboratory equipment 130, and the client device 140 may be communicatively coupled via a network 150.

In some cases, the one or more laboratory equipment 130 may include any wetlab and dry lab equipment capable of synthesis, purification, and/or analysis. Examples of the one or more laboratory equipment 130 may include synthesizers, including standard equipment (e.g., fume hoods, glassware, heating and cooling devices, stirrers), automated and specialized synthesis platforms (e.g., automated synthesizers, parallel synthesis workstations, and high-throughput experimentation (HTE) for efficient reaction optimization, and specialized reactors (e.g., microwave, flow, photochemistry). In some cases, the one or more laboratory equipment 130 may also include tools to support various purification techniques such as chromatography (e.g., silica gel chromatography, preparative high performance liquid chromatography (Prep HPLC)), centrifugal, crystallization/recrystallization, and/or the like. In some cases, the one or more laboratory equipment 130 may also include analytical tools such as microscopes, spectroscopes, spectrometers (e.g., nuclear magnetic resonance (NMR) spectrometers, mass spectrometers), balances, pH meters, elemental analyzers, and/or the like.

In some cases, the client device 140 may be a processor-based device including, for example, a workstation, a desktop computer, a laptop computer, a smartphone, a tablet computer, a wearable apparatus, and/or the like. The network 150 may be a wired network and/or a wireless network including, for example, a local area network (LAN), a virtual local area network (VLAN), a wide area network (WAN), a public land mobile network (PLMN), the Internet, and/or the like.

In some example embodiments, the selection engine 120 may include a property computation model 125 and a selection controller 127. In some cases, the property computation model 125 may be trained to determine one or more biophysical descriptors defining one or more surface properties of one or more molecule designs 116 generated by the molecule design computation model 115. In some cases, the molecule design computation model 115 may be trained to generate the one or more molecule designs 116 to be binders of a target molecule 118 (e.g., a viral antigen, a tumor antigen, and/or the like). However, as noted, to be a viable therapeutic protein, a molecule design may be required to exhibit certain developability traits in addition to exhibiting adequate binding affinity and binding specificity toward the target molecule 118. Accordingly, as described in more detail below, the one or more molecule designs 116 may be screened for developability traits (or developability liabilities). For example, in some cases, one or more biophysical descriptors may be defined to capture one or more surface properties associated with developability. That is, in some cases, the one or more biophysical descriptors may serve as a quantifiable proxy for developability traits (or developability liabilities) such as clearance, solubility, viscosity, storability, expression, manufacturability, polyspecificity (or polyreactivity), pharmacokinetics (PK) (e.g., long in vivo half-life for sustained efficacy), stability (e.g., resistant to heat, pH changes, storage conditions, and/or the like), and/or the like.

In some example embodiments, the one or more biophysical descriptors may define surface properties for electrostatics, hydrophobicity, chemical liabilities, and/or the like. In some cases, the property computation model 125 may be trained to determine, based at least on the amino acid residue sequence of each molecule design 116, the one or more biophysical descriptors. It should be appreciated that the property computation model 125 being sequence-based may enable candidate molecules to be screened for developability liabilities with greater computational efficiency and scalability than conventional structural-based approaches.

In some example embodiments, the one or more molecule designs 116 may be screened for the developability liabilities, for example, by the selection controller 127, based on the one or more biophysical descriptors determined by the property computation model 125. For example, in some cases, the selection controller 127 may select, from the one or more molecule designs 116, at least one candidate molecule 119 to advance to one or more subsequent stages of the drug development pipeline, such as synthesis and experimental validation by the one or more laboratory equipment 130, if the one or more biophysical descriptors of the candidate molecule 119 determined by the property computation model 125 indicate the absence of developability liabilities.

In some example embodiments, instead of individual biophysical descriptors, the one or more molecule designs 116 may be selected based on multiple biophysical descriptors, in which case the selection controller 127 may perform multi-objective optimization including by at least computing a utility metric for each molecule design 116. In some cases, the utility metric of an individual molecule design (e.g., a first molecule design 116a) may quantify the extent to which the biophysical descriptors of the molecule design 116 improve upon those of one or more other molecule designs (e.g., a second molecule design 116b) from the same design iteration (or a baseline candidate molecule from a previous design iteration). In some cases, the utility metric may correspond to the probability of each molecule design 116 being one of the Pareto-optimal solutions populating the Pareto frontier. In some cases, the utility metric of each molecule design 116 may correspond to the distance (or proximity) between each molecule design 116 and the Pareto frontier, meaning that each molecule design 116 may be identified as a Pareto-optimal solution on the Pareto frontier (or a non-Pareto-optimal solution) depending on whether its utility metric satisfies one or more thresholds. Examples of utility metrics include expected hypervolume improvement (EHVI), noisy expected hypervolume improvement (NEHVI), Pareto efficient global optimization (ParEGO), max-value entropy search method (MESMO), joint entropy search (JES), expected multivariate rank, and/or the like. In some cases, the selection controller 127 may select at least the candidate molecule 119, for example, from the molecule designs 116, for advancement to one or more subsequent stages of the drug development pipeline where the utility metric of the candidate molecule 119 satisfies one or more criteria. For instance, in some cases, the one or more criteria may include the utility metric of the candidate molecule 119 satisfying one or more thresholds. Alternatively and/or additionally, the one or more criteria may include the utility metric of the candidate molecule 119 being better than the utility metric of a threshold quantity of other molecule designs from the same and/or previous design iterations.

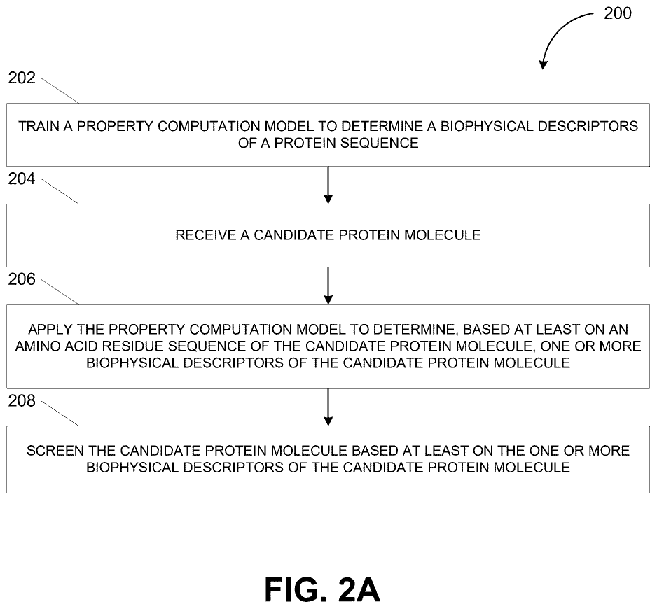

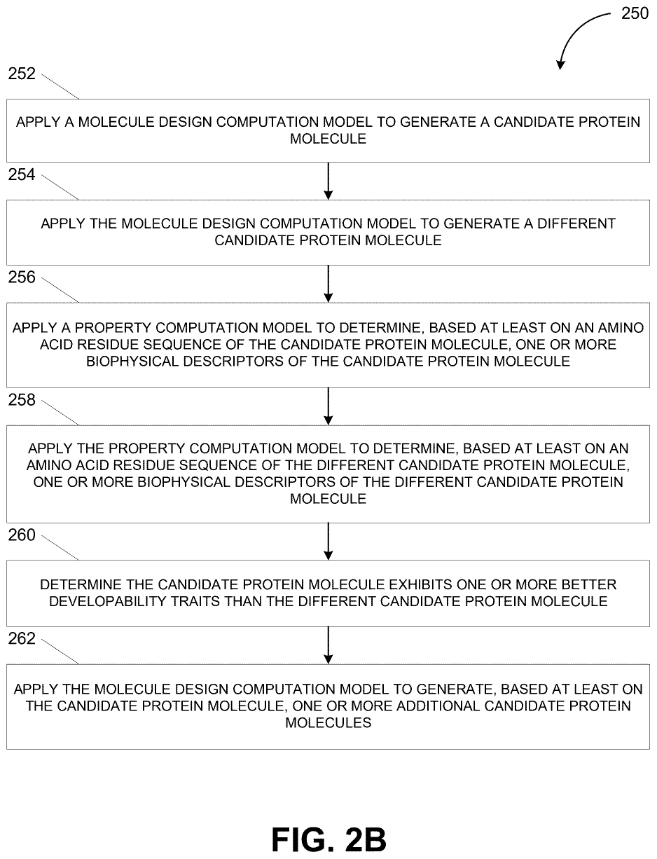

FIG. 2A depicts a flowchart illustrating an example of a process 200 for machine learning enabled design of therapeutic protein molecules with biophysical property optimization, in accordance with some example embodiments. Referring to FIGS. 1 and 2A, in some cases, the process 200 may be performed by the molecule design system 100. For example, in some cases, the molecule design computation model 115 may be applied to generate the one or more molecule designs 116. In some cases, in addition to binding affinity and/or binding specificity toward the target molecule 118, the one or more molecule designs 116 may be screened for developability traits (or developability liabilities) such as clearance, solubility, viscosity, storability, expression, manufacturability, polyspecificity (or polyreactivity), pharmacokinetics (PK) (e.g., long in vivo half-life for sustained efficacy), stability (e.g., resistant to heat, pH changes, storage conditions, and/or the like), and/or the like. For instance, in some cases, the property computation model 125 may be applied to determine, based at least on the amino acid residue sequence of each molecule design 116, one or more biophysical descriptors defining one or more surface properties associated with developability. In some cases, the one or more molecule designs 116 may be screened based at least on the one or more biophysical descriptors. Alternatively and/or additionally, in some cases, the generating of the one or more molecule designs 116 by the molecule design computation model 115 may be guided by the one or more biophysical descriptors such that the molecule design computation model 115 generates molecule designs with incrementally better developability traits. That the property computation model 125 is sequence-based may enable candidate molecules to be screened for developability liabilities with greater computational efficiency and scalability than conventional structure-based approaches.

At 202, a property computation model is trained to determine one or more biophysical descriptors of a protein sequence. In some example embodiments, the property computation model may include one or more machine learning models trained to determine, based at least on an amino acid residue sequence of the protein sequence, the one or more biophysical descriptors. In some cases, the one or more biophysical descriptors may define one or more surface properties including, for example, electrostatics, hydrophobicity, chemical liabilities, and/or the like. In some cases, the one or more surface properties may be associated with developability traits (or developability liabilities) including, for example, clearance, solubility, viscosity, storability, expression, manufacturability, polyspecificity (or polyreactivity), pharmacokinetics (PK) (e.g., long in vivo half-life for sustained efficacy), stability (e.g., resistant to heat, pH changes, storage conditions, and/or the like), and/or the like. In some cases, the property computation model may include one or more machine learning models such as, for example, an encoder (e.g., transformer encoders, convolutional encoders, and/or the like) coupled with one or more regression models. In some cases, the property computation model may include one or more ensembles of machine learning models, each of which including, for example, an encoder coupled with one or more regression models. In some cases, the property models may include multiple ensembles of machine learning models, each of which being trained to determine the biophysical descriptors of a different surface property. For example, in some cases, the property computation model may include one ensemble (e.g., including an encoder coupled with one or more regression models) trained to determine the biophysical descriptors of one surface property (e.g., electrostatics). Furthermore, in some cases, the property computation model may include an additional ensemble (e.g., including an encoder coupled with one or more regression models) trained to determine the biophysical descriptors of a different surface property (e.g., hydrophobicity).

In some example embodiments, the property computation model may be trained based on a training dataset that includes one or more ground-truth surface properties derived from computational modeling of protein structures. For example, in some cases, the property computation model may be trained based on protein structures from the paired Observed Antibody Space (pOAS) that have been folded using physics-based modeling. Furthermore, in some cases, the biophysical descriptors defining the surface properties associated with developability may be benchmarked to establish robust optimization parameters and strengthen the nexus between the biophysical descriptors and experimental data. In some cases, the property computation model may be trained, based on the training dataset, to determine the nexus between the amino acid residue sequence of protein molecules and the biophysical descriptors that are present in these protein molecules. Accordingly, once trained, the sequence-based nature of the property computation model may increase the computational efficiency and scalability of downstream applications applying the property computation model including, for example, the screening of candidate protein molecules, guiding the generating of candidate protein molecules, and/or the like.

At 204, a candidate protein molecule is received. In some example embodiments, receiving the candidate protein molecule may include receiving the amino acid residue sequence of the candidate protein molecule. In some cases, the candidate protein molecule may include at least a portion of a protein molecule, such as an antibody or a portion of the antibody (e.g., the variable region (Fv), the antigen binding fragment (Fab), one or more complementarity determining regions (CDRs), and/or the like). In some cases, the candidate protein molecule may be generated at least part computationally (or in silico) by applying a molecule design computation model (e.g., a diffusion model).

At 206, the property computation model is applied to determine, based at least on an amino acid residue sequence of the candidate protein molecule, one or more biophysical descriptors of the candidate protein molecule. In some example embodiments, once trained, the property computation model may be applied to determine, based at least on the amino acid residue sequence of the candidate protein molecule, the one or more biophysical descriptors defining one or more surface properties associated with the developability of the candidate protein molecule. As noted, in some cases, the one or more biophysical descriptors may define one or more surface properties for electrostatics, hydrophobicity, chemical liabilities, and/or the like. Furthermore, in some cases, the one or more surface properties may approximate one or more corresponding developability traits (or developability liabilities) including, for example, clearance, solubility, viscosity, storability, expression, manufacturability, polyspecificity (or polyreactivity), pharmacokinetics (PK) (e.g., long in vivo half-life for sustained efficacy), stability (e.g., resistant to heat, pH changes, storage conditions, and/or the like), and/or the like.

At 208, the candidate protein molecule is screened based at least on the one or more biophysical descriptors of the candidate protein molecule. In some example embodiments, the candidate protein molecule may be selected, based at least on the one or more biophysical descriptors determined by the property computation model, to advance to one or more subsequent stages of the drug development pipeline. For example, in some cases, the one or more biophysical descriptors may indicate that the candidate protein molecule exhibits satisfactory developability traits (or lacks developability liabilities) such as solubility, stability, viscosity, aggregation, immunogenicity, expression, and/or the like. As such, in some cases, a selection controller may select the candidate protein molecule for synthesis, experimental validation, and other further development efforts. For instance, in some cases, the selection controller may select the candidate protein molecule for further development where the one or more biophysical descriptors of the candidate protein molecule satisfy one or more thresholds. Alternatively, in some cases, the selection controller may select the candidate protein molecule for further development where the candidate protein molecule exhibits better biophysical descriptors than a threshold quantity of other candidate protein molecules generated by the molecule design computation model during the same (or different) design iteration.

In some example embodiments, instead of individual biophysical descriptors, the candidate protein molecule may be screened based on multiple biophysical descriptors. Accordingly, in some cases, the selection controller may perform multi-objective optimization, which may include computing a utility metric for the candidate protein molecule. In some cases, the utility metric of the candidate protein molecule may quantify the extent to which the biophysical descriptors of the candidate protein molecule improve upon those of one or more other candidate protein molecules from the same design iteration (or a baseline candidate protein molecule from a previous design iteration). In some cases, the utility metric may correspond to the probability of the candidate protein molecule being one of the Pareto-optimal solutions populating the Pareto frontier. In some cases, the utility metric of the candidate protein molecule may correspond to the distance (or proximity) between the candidate protein molecule and the Pareto frontier, such that the candidate protein molecule may be identified as a Pareto-optimal solution on the Pareto frontier if its utility metric satisfies one or more thresholds. Examples of utility metrics in this context may include expected hypervolume improvement (EHVI), noisy expected hypervolume improvement (NEHVI), Pareto efficient global optimization (ParEGO), max-value entropy search method (MESMO), joint entropy search (JES), expected multivariate rank, and/or the like. In some cases, the selection controller may select at least the candidate protein molecule for advancement to one or more subsequent stages of the drug development pipeline where the utility metric of the candidate protein molecule satisfies one or more criteria. For instance, in some cases, the one or more criteria may include the utility metric of the candidate protein molecule satisfying one or more thresholds. Alternatively and/or additionally, the one or more criteria may include the utility metric of the candidate protein molecule being better than the utility metric of a threshold quantity of other candidate protein molecules from the same and/or previous design iterations.

FIG. 2B depicts a flowchart illustrating an example of a process 250 for machine learning enabled design of therapeutic protein molecules with biophysical property optimization, in accordance with some example embodiments. Referring to FIGS. 1 and 2B, in some cases, the process 250 may be performed by the molecule design system 100. For example, in some cases, the molecule design computation model 115 may be applied to generate the one or more molecule designs 116. In some cases, the generating of the one or more molecule designs 116 may be guided by the biophysical descriptors determined by the property computation model 125. For instance, in some cases, the property computation model 125 may be applied to determine, based at least on the amino acid residue sequence of each molecule design 116, the one or more biophysical descriptors. In some cases, the one or more biophysical descriptors may define one or more surface properties associated with developability traits (or developability liabilities) such as clearance, solubility, viscosity, storability, expression, manufacturability, polyspecificity (or polyreactivity), pharmacokinetics (PK) (e.g., long in vivo half-life for sustained efficacy), stability (e.g., resistant to heat, pH changes, storage conditions, and/or the like), and/or the like. In some cases, guidance based on by the one or more biophysical descriptors may enable the molecule design computation model 115 to generate molecule designs with incrementally better developability traits. As noted, the sequence-based nature of the property computation model 125 may increase the computational efficiency and scalability of developability guided protein generation relative to conventional structural-based approaches.

At 252, a molecule design computation model is applied to generate a candidate protein molecule. In some example embodiments, the molecule design computation model may include one or more machine learning models, such as a diffusion model, that has been trained to generate the candidate protein molecule by at least modifying an input protein sequence. For example, in some cases, the molecule design computation model may generate the candidate protein molecule by inserting, deleting, and/or changing an identity (or type) of one or more amino acid residues in the input protein sequence. As described in more detail below, in some cases, the molecule design computation model may modify the input protein sequence incrementally, while guided by the biophysical descriptors of the modified protein sequences.

At 254, the molecule design computation model is applied to generate a different candidate protein molecule. In some example embodiments, the molecule design computation model may be applied to modify the input protein sequence and generate multiple modified protein sequences. For example, in some cases, the molecule design computation model may generate the candidate protein molecule by inserting, deleting, and/or changing an identity (or type) of one or more amino acid residues in the input protein sequence. In some cases, in doing so, the molecule design computation model may generate an additional candidate protein molecule having a different amino acid residue sequence than the candidate protein molecule generated in operation 252. As described in more detail below, in some cases, the molecule design computation model may modify the input protein sequence incrementally, while guided by the biophysical descriptors of the modified protein sequences.

At 256, a property computation model is applied to determine, based at least on an amino acid residue sequence of the candidate protein molecule, one or more biophysical descriptors of the candidate protein molecule. In some example embodiments, the property computation model may include one or more machine learning models trained to determine, based at least on an amino acid residue sequence of the protein sequence, the one or more biophysical descriptors. For example, in some cases, the property computation model may include one or more ensembles of machine learning models (e.g., an encoder coupled with one or more regression models), each of which being trained to determine the biophysical descriptors of a different surface property such as electrostatics, hydrophobicity, chemical liabilities, and/or the like. In some cases, the one or more surface properties may be associated with developability traits (or developability liabilities) including, for example, clearance, solubility, viscosity, storability, expression, manufacturability, polyspecificity (or polyreactivity), pharmacokinetics (PK) (e.g., long in vivo half-life for sustained efficacy), stability (e.g., resistant to heat, pH changes, storage conditions, and/or the like), and/or the like.

At 258, the property computation model is applied to determine, based at least on an amino acid residue sequence of the different candidate protein molecule, one or more biophysical descriptors of the different candidate protein molecule. In some example embodiments, the property computation model may also be applied to determine, based at least on an amino acid residue sequence of the different candidate protein molecule, one or more biophysical descriptors defining one or more surface properties associated with developability. In some cases, the one or more biophysical descriptors may define surface properties for electrostatics, hydrophobicity, chemical liabilities, and/or the like. In some cases, the one or more surface properties may approximate developability traits (or developability liabilities) such as clearance, solubility, viscosity, storability, expression, manufacturability, polyspecificity (or polyreactivity), pharmacokinetics (PK) (e.g., long in vivo half-life for sustained efficacy), stability (e.g., resistant to heat, pH changes, storage conditions, and/or the like), and/or the like.

At 260, the candidate protein molecule is determined to exhibit one or more better developability traits than the different candidate protein molecule. In some example embodiments, a selection controller may determine, based at least on the one or more biophysical descriptors of each candidate protein molecule, that the candidate protein molecule exhibits better developability traits (or fewer developability liabilities) than the different candidate protein molecule. In some cases, the developability traits (or developability liabilities) of two (or more) candidate protein molecules may be assessed based on multiple biophysical descriptors. For instance, where two (or more) candidate protein molecules are assessed based on multiple biophysical descriptors, the selection controller may perform multi-objective optimization by at least computing a utility metric for each candidate protein molecule. In some cases, the utility metric of a candidate protein molecule may quantify the extent to which the biophysical descriptors of the candidate protein molecule improve upon those of one or more other candidate protein molecules from the same design iteration (or a baseline candidate protein molecule from a previous design iteration). In some cases, the utility metric may correspond to the probability of a candidate protein molecule being one of the Pareto-optimal solutions populating the Pareto frontier. In some cases, the utility metric of a candidate protein molecule may correspond to the distance (or proximity) between the candidate protein molecule and the Pareto frontier, such that the candidate protein molecule may be identified as a Pareto-optimal solution on the Pareto frontier if its utility metric satisfies one or more thresholds. Examples of utility metrics in this context may include expected hypervolume improvement (EHVI), noisy expected hypervolume improvement (NEHVI), Pareto efficient global optimization (ParEGO), max-value entropy search method (MESMO), joint entropy search (JES), expected multivariate rank, and/or the like.

At 262, the molecule design computation model is applied to generate, based at least on the candidate protein molecule, one or more additional candidate protein molecules. In some example embodiments, the molecule design computation model may be applied to modify the input protein sequence, for example, by inserting, deleting, and/or changing an identity (or type) of one or more constituent amino acid residues, to incrementally improve its developability traits (or developability liabilities). For example, in some cases, the selection controller may select at least the candidate protein molecule for further modification by the molecule design computation model based at least on the one or more biophysical descriptors of the candidate protein molecule. In some cases, the selection controller may select the candidate protein molecule for further modification instead of the different candidate protein molecule based at least on the biophysical properties of the candidate protein molecule indicating that the candidate protein molecule exhibits better developability traits (or fewer developability liabilities) than the different candidate protein molecule. For instance, in some cases, the molecule design computation model may be applied to insert, delete, and/or change an identity (or type) of one or more amino acid residues forming the candidate protein molecule. In doing so, the molecule design computation model may generate one or more additional candidate protein sequences. In some cases, the one or more additional candidate protein sequences may be assessed based on the corresponding developability traits (or developability liabilities), as approximated by the surface properties defined by the biophysical descriptors determined by the property computation model.

In some example embodiments, the molecule design computation model may be applied to continue modifying the input protein sequence until one or more criteria are satisfied. For example, in some cases, the molecule design computation model may be applied to continue modifying the input protein sequence until one or more developability traits (or developability liabilities) of the resulting candidate protein sequence satisfy one or more thresholds. Alternatively and/or additionally, the molecule design computation model may be applied to continue modifying the input protein sequence until a threshold quantity of candidate protein sequences are generated. In some cases, the molecule design computation model may be applied to continue modifying the input protein sequence until a threshold quantity of candidate protein sequences whose developability traits (or developability liabilities) satisfy one or more thresholds are generated.

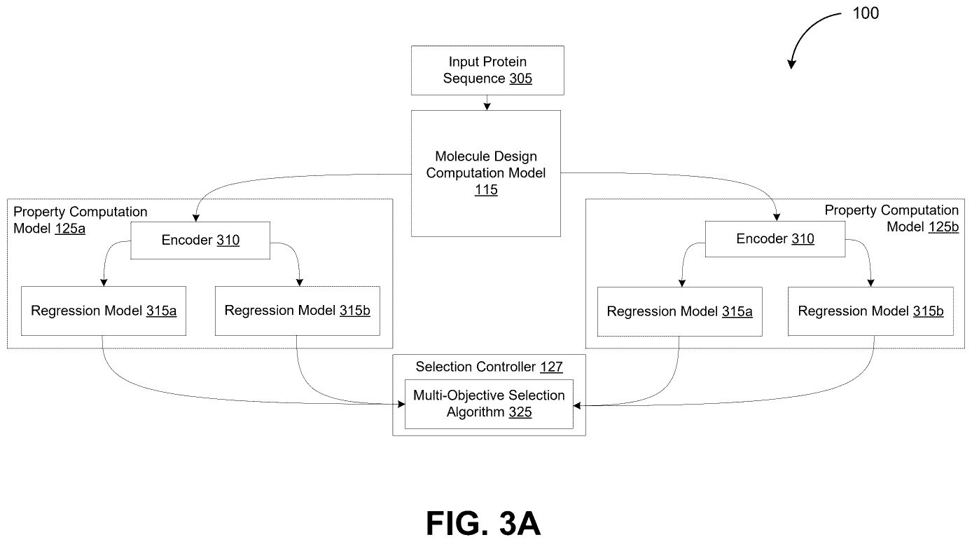

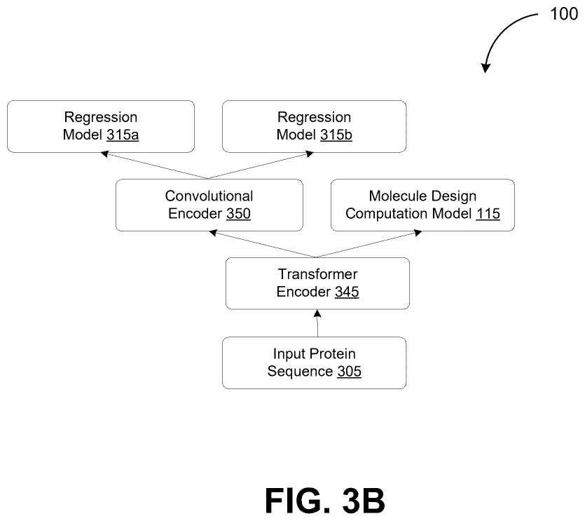

FIG. 3A depicts a schematic diagram illustrating an example deployment of the molecule design system 100, in accordance with some example embodiments. Referring to FIGS. 1 and 3A, in some example embodiments, the property computation models 125 may include one or more machine learning models, such as ensembles of encoders (e.g., transformer encoders, convolutional encoders, and/or the like) coupled with regression models, trained to determine, based at least on the amino acid residue sequence of the one or more molecule designs 116 generated by the molecule design computation model 115, one or more biophysical descriptors defining one or more surface properties associated with developability. In the example shown in FIG. 3A, the molecule design computation model 115 may generate the one or more molecule designs 116 by at least modifying an input protein sequence 305 including by, for example, inserting, deleting, and/or changing an identity (or type) of one or more constituent amino acid residues.

Referring again to FIG. 3A, in the example deployment shown therein, the selection engine 120 may include multiple instantiations of the property computation model 125. In some cases, each instance of property computation model 125 may include an ensemble of an encoder 310 coupled with one or more regression models 315 (e.g., a first regression model 315a, a second regression model 315b, and/or the like). For example, FIG. 3B shows an example in which the property computation model 125 includes a transformer encoder 345 coupled with a convolutional encoder 350 that is then further coupled with the first regression model 315a and the second regression model 315b. In some cases, each instance of property computation model 125 may be instantiated with different weights and trained to determine one or more different biophysical descriptors (e.g., defining one or more surface properties) of the input protein sequence 305 than the other instances of the property computation model 125. For instance, in some cases, the first instance of property computation model 125a may be trained to determine one or more electrostatic properties of the input protein sequence 305 while the second instance of property computation model 125b may be trained to determine the hydrophobicity of the input protein sequence 305. In some cases, the selection controller 127 may apply select, based at least on the biophysical descriptors determined by the one or more instances of the property computation model 125, a subset of the molecule designs 116 to advance to one or more subsequent stages of the drug development pipeline, such as synthesis, experimental validation, and/or the like. In instances where the molecule designs 116 are evaluated on multiple biophysical descriptors, the selection controller 127 may apply a multi-objective selection algorithm 325, which may include selecting the subset of the molecules designs 116 based on the utility metric of each molecule design.

In some example embodiments, the generating of the molecule designs 116 by the molecule design computation model 115 may be guided by the biophysical descriptors determined by the one or more instances of the property computation model 125. For example, in some cases, the modifying of the input protein sequence 305 by the molecule design computation model 115 may be guided by the electrostatic properties determined by the first instance of property computation model 125a and the hydrophobicity determined by the second instance of property computation model 125b. In some cases, guidance from the biophysical descriptors determined by the property computation model 125 may enable the molecule design computation model 115 to generate protein sequences (or amino acid residue sequences) whose surface properties are associated with incrementally improved developability traits. Examples of developability traits include solubility, stability, viscosity, aggregation, immunogenicity, expression, and/or the like. In some cases, improved developability traits may increase the likelihood of a protein sequence (e.g., a computationally designed protein sequence) becoming a viable protein therapeutic. For instance, in some cases, the molecule design computation model 115 may be applied to modify the input protein sequence 305 by at least inserting, deleting, and/or changing the identity (or type) of one or more constituent amino acid residues. In some cases, the input protein sequence 305 may be modified to generate at least the first molecule design 116a and the second molecule design 116b. In some cases, the property computation model 125 may be applied to determine, for each of the first molecule design 116a and the second molecule design 116b, one or more corresponding biophysical descriptors. In some cases, the molecule design computation model 125 may be applied to further modify the first molecule design 116a instead of the second molecule design 116b based at least on the biophysical descriptors of the first molecule design 116a being associated with better developability traits than the biophysical descriptors of the second molecule design 116b.

### Experimental Examples

Various examples of the property computation model 125 described herein were trained to predict APBS electrostatics and SAP hydrophobicity directly from the amino acid residue sequence of a protein molecule. A synthetic biophysical dataset was constructed by computing physics-based descriptors over 1 million antibody sequences folded using ESMFold from the paired Observed Antibody Space (pOAS) clustered at 95% sequence identity. Given the minor effects from structure preparation, sequence diversity was prioritized over structural accuracy by training on descriptors computed over static antibody binding fragment (Fab) structures. The property computation model 125 was trained using an 80/20 train/test split. The Spearman's (or rank) correlation of descriptors inferred from the property computation model 125 with those computed from physics-based models approaches 0.9 over a held out test set sampled independently and identically distributed from the pOAS (N=99118). When compared to physics-based descriptors on viscosity datasets, the resulting Spearman's (or rank) correlations remain close to 0.9 for global variants such as those in Ab21, and range be-tween 0.5-0.9 when variants are a few point mutations from the parent molecule such as in GCGR and PDGF38, although 0.9 metrics was also achieved on for the mechanistically relevant surface properties. When employed as a filter across all viscosity datasets, the property computation model 125 yields a substantially lower FPR of 0.07 and a similar FNR of 0.25 for both unusual or problematic viscosity (e.g., amber and red flags), compared to more computationally expensive physics-based approaches.

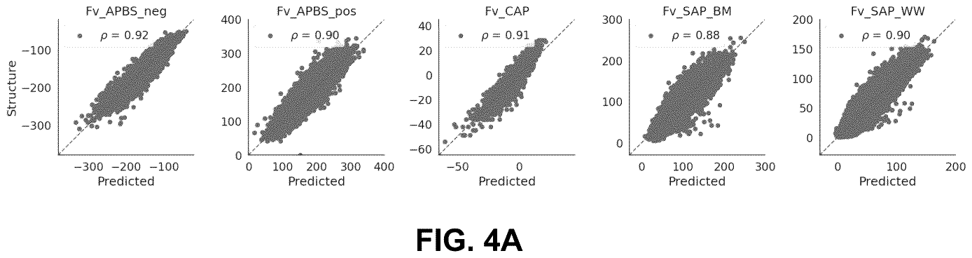

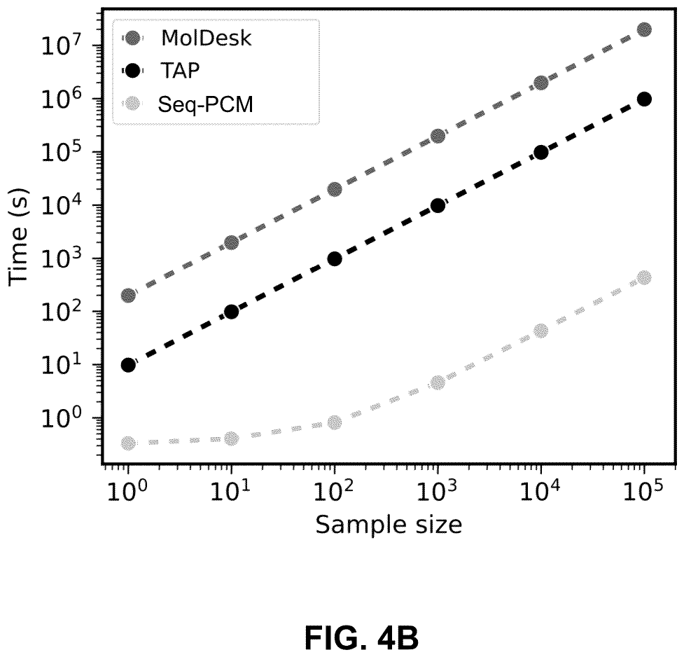

FIG. 4A depicts graphs illustrating a comparison of various structurally determined surface properties and the corresponding surface properties determined using various example embodiments of the sequence-based property computation model 125 described herein. Specifically, FIG. 4A depicts the Spearman's (or rank) correlation p between the structurally determined surface properties and the same surface properties determined using the property computation model 125. The surface properties shown in FIG. 4A include, within the variable region (Fv) of an antibody, regions of negative electrostatic potential (Fv_APBS_neg), regions of negative electrostatic potential (Fv_APBS pos), charge and polarity (Fv_CAP), Black and Mould scale hydrophobicity (Fv_SAP_BM), and Wimely-White scale hydrophobicity (Fv_SAP_WW). As shown in FIG. 4A, the biophysical descriptors determined by the property computation model 125 correlates strongly with the corresponding structurally predicted surface properties (e.g., Spearman's (or rank) correlation p between 0.88 and 0.92).

FIG. 4B depicts a graph illustrating the computational scalability of various example embodiments of the sequence-based property computation model 125 described herein. As noted, that the property computation model 125 is sequence based means that the property computation model 125 is more computationally efficient and scalable than conventional structural-based solutions. As shown in FIG. 4B, the property computation model 125 (denoted Seq-PCM in FIG. 4B) consumes less GPU time than conventional solutions MolDesk and Therapeutic Antibody Profiler (TAP) for all sample sizes.

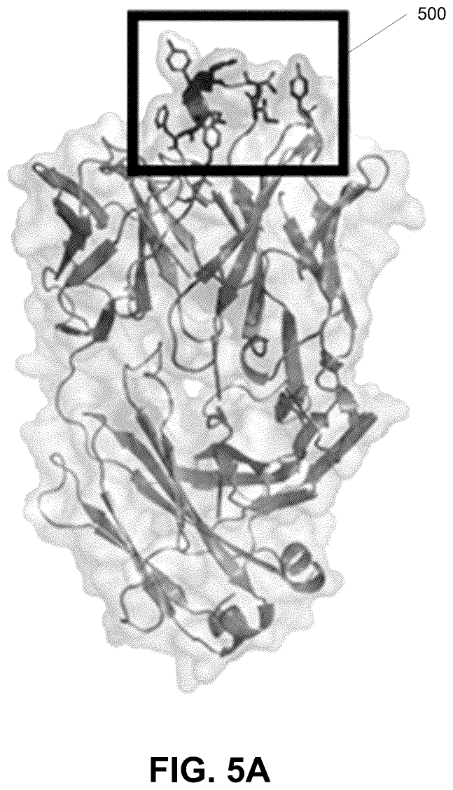

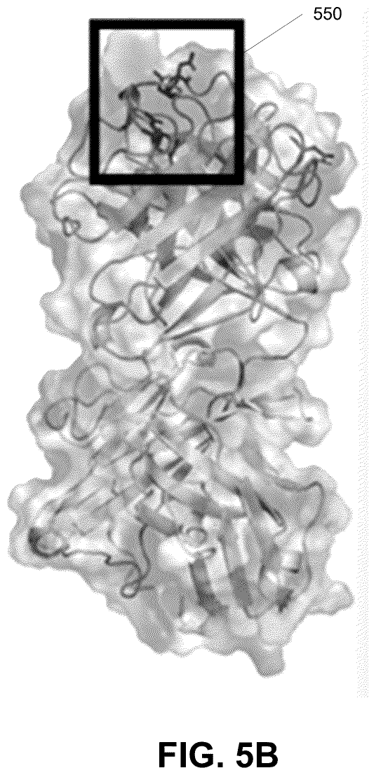

The property computation model 125 was also used to guide the generation of novel protein sequences using diffusion optimized sampling (NOS) within a multi-objective Bayesian optimization framework. Edits within the starting sequence are chosen using feature attributions to greedily determine the most important positions. Guidance from the property computation model 125 offers a flexible solution, as antibody properties can be mechanistically driven by different biophysical interactions. Proof-of-concept was demonstrated with designs around the parental GCGR and PDGF38 molecules, which exhibit a hydrophobicity risk flag and an electrostatic risk flag, respectively. Because guidance from the property computation model 125 modifies protein molecules to be more similar to clinically viable therapeutics, the resulting set of designs for GCGR were improved towards a reduction of SAP scores. Most of the resulting designs target primarily aromatic residues around the hydrophobic patch in the CDRs, such as tryptophan and tyrosine (e.g., box 500 in FIG. 5A). Fourteen proposed designs overlapped with the original experimental dataset, which includes primarily single point mutations. Twelve of the proposed designs decreased viscosity at 180 mg/ml relative to the parent and 9 were below the 30 cP threshold.

Likewise, to mitigate the electrostatic risk flag, guidance from the property computation model 125 improved PDGF38 towards reducing regions of negative electrostatic potential (Fv_APBS_neg). There were no overlap between the proposed designs and the experimental set, partly because the reference dataset sampled higher ranges of mutations. Nevertheless, many mutations also appear to primarily target the electronegative patch in the variable region (e.g., box 550 in FIG. 5B).

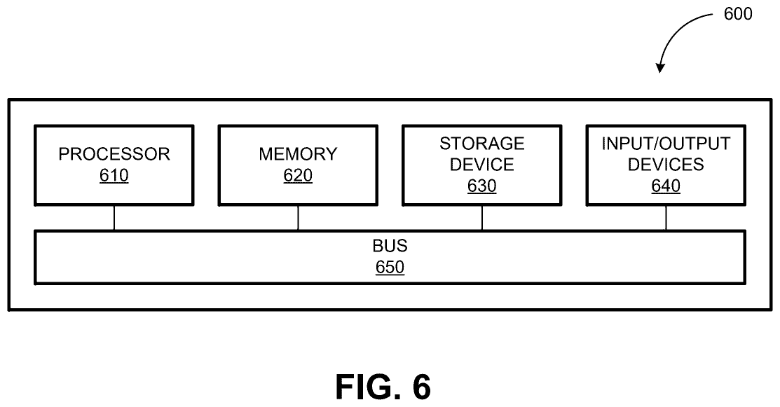

FIG. 6 depicts a block diagram illustrating an example of a computing system 600, in accordance with some example embodiments. Referring to FIGS. 1-6, the computing system 600 may be used to implement the molecule design engine 110, the selection engine 120, the client device 130, and/or any components therein.

As shown in FIG. 6, the computing system 600 can include a processor 610, a memory 620, a storage device 630, and input/output devices 640. The processor 610, the memory 620, the storage device 630, and the input/output devices 640 can be interconnected via a system bus 650. The processor 610 is capable of processing instructions for execution within the computing system 600. Such executed instructions can implement one or more components of, for example, the molecule design engine 110, the selection engine 120, the client device 130, and/or the like. In some example embodiments, the processor 610 can be a single-threaded processor. Alternately, the processor 610 can be a multi-threaded processor. The processor 610 is capable of processing instructions stored in the memory 620 and/or on the storage device 630 to display graphical information for a user interface provided via the input/output device 640.

The memory 620 is a computer readable medium such as volatile or non-volatile that stores information within the computing system 600. The memory 620 can store data structures representing configuration object databases, for example. The storage device 630 is capable of providing persistent storage for the computing system 600. The storage device 630 can be a floppy disk device, a hard disk device, an optical disk device, or a tape device, or other suitable persistent storage means. The input/output device 640 provides input/output operations for the computing system 600. In some example embodiments, the input/output device 640 includes a keyboard and/or pointing device. In various implementations, the input/output device 640 includes a display unit for displaying graphical user interfaces.

According to some example embodiments, the input/output device 640 can provide input/output operations for a network device. For example, the input/output device 640 can include Ethernet ports or other networking ports to communicate with one or more wired and/or wireless networks (e.g., a local area network (LAN), a wide area network (WAN), the Internet).

In some example embodiments, the computing system 600 can be used to execute various interactive computer software applications that can be used for organization, analysis and/or storage of data in various formats. Alternatively, the computing system 600 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 640. The user interface can be generated and presented to a user by the computing system 600 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT), a liquid crystal display (LCD), a light emitting diode (LED) monitor, or an organic light emitting diode (OLED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C,” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A computer-implemented method, comprising:
receiving a candidate protein molecule, wherein the candidate protein molecule comprises an amino acid residue sequence;
applying a property computation model to determine, based at least on the amino acid residue sequence of the candidate protein molecule, one or more biophysical descriptors of the candidate protein molecule, wherein the one or more biophysical descriptors define one or more surface properties of the candidate protein molecule; and
screening the candidate protein molecule based at least on the one or more biophysical descriptors of the candidate protein molecule.

2. The method of claim 1, wherein the one or more biophysical descriptors define the one or more surface properties for electrostatics, hydrophobicity, and/or chemical liabilities.

3. The method of any of claims 1 to 2, wherein the one or more surface properties approximate one or more developability traits.

4. The method of claim 3, wherein the one or more developability traits include clearance, solubility, viscosity, storability, expression, manufacturability, polyspecificity, polyreactivity, pharmacokinetics (PK), and stability.

5. The method of any of claims 1 to 4, wherein the property computation model includes one or more machine learning models.

6. The method of any of claims 1 to 5, wherein the property computation model includes one or more ensembles of machine learning models.

7. The method of claim 6, wherein each ensemble of machine learning models includes an encoder coupled with one or more regression models.

8. The method of any of claims 6 to 7, wherein each ensemble of machine learning models includes a transformer encoder coupled with a convolutional encoder that is further coupled with one or more regression models.

9. The method of any of claims 6 to 8, wherein each ensemble of machine learning models is trained to determine an individual biophysical descriptor.

10. The method of any of claims 6 to 9, wherein the one or more ensembles of machine learning models includes one ensemble trained to determine a biophysical descriptor defining an electrostatic surface property.

11. The method of claim 10, wherein the one or more ensembles of machine learning models includes an additional ensemble trained to determine a biophysical descriptor defining a hydrophobicity surface property.

12. The method of any of claims 1 to 11, wherein the candidate protein molecule is screened based at least on whether the one or more biophysical descriptors of the candidate protein molecule satisfy one or more criteria.

13. The method of any of claims 1 to 12, wherein the candidate protein molecule is screened based on a plurality of biophysical descriptors.

14. The method of claim 13, wherein the screening the candidate protein molecule includes determining a utility metric quantifying an extent to which the plurality of biophysical descriptors of the candidate protein molecules improve upon a same plurality of biophysical descriptors of one or more other candidate protein molecules.

15. The method of claim 14, wherein the utility metric comprises one or more of an expected hypervolume improvement (EHVI), noisy expected hypervolume improvement (NEHVI), Pareto efficient global optimization (ParEGO), max-value entropy search method (MESMO), joint entropy search (JES), and expected multivariate rank.

16. The method of any of claims 1 to 15, wherein the screening the candidate protein molecule includes selecting the candidate protein molecule for synthesis and/or experimental validation.

17. The method of any of claims 1 to 16, wherein the screening the candidate protein molecule includes further modifying the amino acid residue sequence of the candidate protein molecule to generate one or more additional candidate protein molecules.

18. The method of any of claims 1 to 17, wherein a molecule design computation model is applied to further modify the amino acid residue sequence of the candidate protein molecule while guided by the one or more biophysical descriptors.

19. A system, comprising:
at least one data processor; and
at least one memory storing instructions, which when executed by the at least one data processor, result in operations comprising the method of any of claims 1 to 18.

20. A non-transitory computer readable medium storing instructions, which when executed by the at least one data processor, result in operations comprising the method of any of claims 1 to 18.

21. A computer-implemented method, comprising:
applying a molecule design computation model to generate a candidate protein molecule;
applying the molecule design computation model to generate a different candidate protein molecule;
applying a property computation model to determine, based at least on an amino acid residue sequence of the candidate protein molecule, one or more biophysical descriptors defining one or more surface properties of the candidate protein molecule, wherein the one or more surface properties of the candidate protein molecule approximate one or more developability traits of the candidate protein molecule;
applying the property computation model to determine, based at least on an amino acid residue sequence of the different candidate protein molecule, one or more biophysical descriptors defining one or more surface properties of the different protein molecule, wherein the one or more surface properties of the different candidate protein molecule approximate one or more developability traits of the different candidate protein molecule;
determining the candidate protein molecule exhibits one or more better developability traits than the different candidate protein molecule; and
applying the molecule design computation model to generate, based at least on the candidate protein molecule, one or more additional candidate protein molecules.

22. The method of claim 21, wherein the molecule design computation model generates the one or more additional candidate protein molecules by modifying the amino acid residue sequence of the candidate protein molecule.

23. The method of any of claims 21 to 22, wherein the one or more biophysical descriptors define one or more surface properties for electrostatics, hydrophobicity, and/or chemical liabilities.

24. The method of any of claims 21 to 23, wherein the one or more developability traits include clearance, solubility, viscosity, storability, expression, manufacturability, polyspecificity, polyreactivity, pharmacokinetics (PK), and stability.

25. The method of any of claims 21 to 24, wherein the property computation model includes one or more machine learning models.

26. The method of any of claims 21 to 25, wherein the property computation model includes one or more ensembles of machine learning models.

27. The method of claim 26, wherein each ensemble of machine learning models includes an encoder coupled with one or more regression models.

28. The method of any of claims 26 to 27, wherein each ensemble of machine learning models includes a transformer encoder coupled with a convolutional encoder that is further coupled with one or more regression models.

29. The method of any of claims 26 to 28, wherein each ensemble of machine learning models is trained to determine an individual biophysical descriptor.

30. The method of any of claims 26 to 29, wherein the one or more ensembles of machine learning models includes one ensemble trained to determine a biophysical descriptor defining an electrostatic surface property.

31. The method of claim 30, wherein the one or more ensembles of machine learning models includes an additional ensemble trained to determine a biophysical descriptor defining a hydrophobicity surface property.

32. The method of any of claims 21 to 31, wherein the candidate protein molecule is determined to exhibit the one or more better developability traits than the different candidate protein molecule based at least on a plurality of biophysical descriptors.

33. The method of claim 32, further comprising:
determining a utility metric quantifying an extent to which the plurality of biophysical descriptors of the candidate protein molecules improve upon a same plurality of biophysical descriptors of the different candidate protein molecules.

34. The method of claim 33, wherein the utility metric comprises one or more of an expected hypervolume improvement (EHVI), noisy expected hypervolume improvement (NEHVI), Pareto efficient global optimization (ParEGO), max-value entropy search method (MESMO), joint entropy search (JES), and expected multivariate rank.

35. The method of any of claims 33 to 34, further comprising:
selecting, based at least on the utility metric of the candidate protein molecule satisfying one or more criteria, the candidate protein molecule for synthesis and/or experimental validation.

36. The method of any of claims 21 to 35, wherein the candidate protein molecule and the different candidate protein molecule each comprise at least a portion of an antibody.

37. The method of any of claims 21 to 36, wherein the candidate protein molecule and the different candidate protein molecule each comprise a variable region (Fv), an antigen binding fragment (Fab), and/or a complementarity determining region (CDR) of an antibody.

38. The method of any of claims 21 to 37, wherein the molecule design computation model is applied to continue modifying the candidate protein molecule and incrementally improve the one or more biophysical descriptors of the candidate protein molecule until one or more criteria are satisfied.

39. A system, comprising:
at least one data processor; and
at least one memory storing instructions, which when executed by the at least one data processor, result in operations comprising the method of any of claims 21 to 38.

40. A non-transitory computer readable medium storing instructions, which when executed by the at least one data processor, result in operations comprising the method of any of claims 21 to 38.
